(19) United States
(12) Patent Application Publication
(10) Pub. No.: US 2025/0228456 A1
(43) Pub. Date: Jul. 17, 2025

(54) Time-Series Anomaly Detection for Healthcare

(71) Applicant: PROTECTING BRAINS & SAVING FUTURES SISTEMA DE DIAGNOSTICOS INTEGRADOS LTDA., Sao Paulo (BR)

(72) Inventor: Gabriel Variane, Sao Paulo (BR)

(21) Appl. No.: 19/169,807

(22) Filed: Apr. 3, 2025

Related U.S. Application Data
(63) Continuation-in-part of application No. PCT/IB2022/059579, filed on Oct. 6, 2022.
(60) Provisional application No. 63/768,401, filed on Mar. 7, 2025.

(51) Int. Cl. A61B 5/00; G16H 10/60; G16H 50/30; G16H 50/70

(52) U.S. Cl. CPC A61B 5/0002; A61B 5/4064; A61B 5/4094; A61B 5/7267; A61B 5/7275; A61B 5/742; A61B 5/746; A61B 5/7475; G16H 10/60; G16H 50/30; G16H 50/70; A61B 2503/04

(57) ABSTRACT

A method or a system for multi-modality infant monitoring. A server receives data associated with a plurality of sets of clinical measurements of an infant in near real time. The infant is located at one of one or more infant care facilities that are remote from the server. The server aggregates the data associated with the plurality of sets of clinical measurements of the infant for a period of time. The server then generates a graphical user interface (GUI) displaying the aggregated data associated with the plurality of sets of clinical measurements of the infant simultaneously, and causes the GUI to be presented on a client device at a monitoring facility, which is also geographically remote from the infant care facility.

### CROSS-REFERENCE TO RELATED APPLICATIONS

This application claims the benefit of U.S. Provisional Application No. 63/768,401, filed on Mar. 7, 2025. This application is also a continuation-in-part of the PCT/IB2022/059579, filed on Oct. 6, 2022. All of the aforementioned applications are incorporated by reference herein in their entirety for all purposes.

### BACKGROUND

Global statistics show over 130 million live births each year, among which 15% of the infants require intensive care unit (ICU) admission. 10-25% of the admitted infants in the ICU are at high risk for brain injury, and over 1,000,000 neonates develop brain injury and disabilities every year worldwide. Even in developed countries, such as the United States, only a small percentage of neonatal intensive care units (NICUs) are capable of providing quality neurocritical care. Infants who develop disabilities due to brain injuries result in devastating social and economic burden on the families and the society.

Studies estimate that in the U.S., the life cost for children who evolve with disabilities is around $67 billions, and that a child with disabilities will have direct health costs up to 150 times higher than a child without disabilities. Similarly, in 2018, indirect costs with a single benefit for people with disabilities were greater than $40 billion in Brazil, which is the third largest governmental expenditure in Brazil. In particular, children represented the highest percentage among different age groups.

This is because children without disabilities only need regular checkups from pediatricians, some childcare consultations, and rare hospitalizations, while children with severe disabilities will require various care from many additional medical specialists, such as neurologists, neurosurgeries, orthopedists, pediatric surgeries, otolaryngologists, ophthalmologists, dentists, physiotherapies, occupational therapists, speech therapies, special medications, ICU admissions, etc.

Brain injuries in infants cause many children's disabilities. Early detection of potential brain injuries would reduce such disabilities in children. Thus, there is a strong interest in reducing brain injuries in infants. However, outside of large medical centers (even in big cities and/or developed countries), sufficient care is often not available due to a lack of monitoring equipment, poor integration of monitoring data, and/or lack of multispecialty expertise in neonatal brain care. The time-critical nature of brain injury, long distances, and suboptimal infrastructure limits the ability and effectiveness of transporting infants to tertiary care centers. Further, developing in-house expertise in outlying NICUs is cost-prohibitive.

### BRIEF DESCRIPTION OF THE DRAWINGS

FIG. 1 is a block diagram of a system environment in which a remote monitoring server operates.

FIG. 2 is a block diagram illustrating an example architecture of the remote monitoring server.

FIG. 3 is a block diagram illustrating an environment in which a local monitoring system 116 resides.

FIGS. 4A and 4B illustrate example GUIs displaying aggregated monitoring data of an infant.

FIG. 5 illustrates an example GUI displaying monitoring data of multiple infants simultaneously.

FIG. 6 illustrates an example architecture of the software, enabling the collection of monitoring data and providing a responsive web interface.

FIG. 7 illustrates an example GUI allowing users to log in.

FIG. 8 illustrates an example GUI displaying a list of monitored patients.

FIG. 9 illustrates an example GUI that shows the control panel.

FIG. 10 is an example GUI showing an example card view of the control panel.

FIG. 11 is an example GUI displaying data associated with NIRs and vital signs.

FIG. 12 is an example GUI displaying data associated with aEEG and raw EEG.

FIG. 13 is an example GUI illustrating a neonatal seizure moment.

FIG. 14 illustrates an example learning framework for training the machine learning model for detecting neonatal seizure moments.

FIG. 15 is an example GUI illustrating tracing of a preterm infant with 25 weeks of gestational age in the second day of life with compromised autoregulation.

FIG. 16 is an example GUI illustrating tracing of a preterm infant, with 25 weeks of gestational age in the third day of life with intact autoregulation.

FIG. 17 is an example GUI illustrating tracing of a preterm infant, with 23 weeks of gestational age in the second day of life with compromised autoregulation.

FIG. 18 is an example GUI displaying trendlines of three score values, including the overall scores, the automatic scores, and the clinic scores.

FIG. 19 is a flowchart of a method of multi-modality monitoring of infants.

FIG. 20 is an example architecture of a machine learning neural network.

FIG. 21 is a block diagram illustrating components of an example computing machine that is capable of reading instructions from a computer-readable medium and executing them in a processor (or controller).

The figures depict embodiments of the present disclosure for purposes of illustration only. One skilled in the art will readily recognize from the following description that alternative embodiments of the structures and methods illustrated herein may be employed without departing from the principles, or benefits touted, of the disclosure described herein.

### DETAILED DESCRIPTION

Several conditions imply risks for brain injury in the neonatal period. The neonatal period has the highest incidence of seizures. High seizure burden leads to permanent brain injury. More than 80% of seizures are completely subclinical. Treating subclinical seizures is associated with less brain injury. However, nonspecific movements may look like seizures, but in fact, they are not. Performing brain monitoring significantly improves seizure detection accuracy, reduces the use of anticonvulsants, and is associated with better neurological outcomes in randomized clinical trials.

Notably, even if the infants are measured with proper equipment, brain injuries of infants are still often overlooked due to poor integration of monitoring data, and/or lack of multispecialty expertise in neonatal brain care. The time-critical nature of brain injury, long distances, and suboptimal infrastructure limits the ability and effectiveness of transporting infants to tertiary care centers, and developing in-house expertise in outlying NICUs is cost prohibitive.

In some embodiments, an advanced telehealth system for improving neurocritical care combining remote multi-modality monitoring and longitudinal education is described. In particular, the principles described herein use a telemedicine system and bioinformatic technologies to connect distant infant care centers to experts (who may be located at a monitoring center or any remote location) to provide real-time specialized monitoring and assistance.

Data associated with clinical measurements of infants from different infant care centers is transmitted to a server, which aggregates the data and presents the aggregated data in a graphical user interface (GUI) on a client device at a central monitoring center (where experts reside) and/or a client device of an expert, who may be located at a work location (e.g., an office) or home. These experts are highly specialized and capable of providing live assistance in real-time 24 hours a day. Real-time may be within a second, a few seconds, a minute, a few minutes, or any time frame that is sufficiently short for diagnosis purposes. These experts can also provide teaching and longitudinal training to personnel at the infant care centers. Such training may be high-level, and/or target a particular infant patient or a particular type of infant patient. The telemedicine system with bioinformatic technologies and the experts facilitate the implementation of methodologies and protocols in different infant care centers with better care and lower costs.

In some embodiments, the experts remotely monitor data and detect anomalies in status that call for specific interventions. In some embodiments, the telemedicine system is configured to train a machine-learning model based on historical infant monitoring data. In some embodiments, the machine learning model is trained using an unsupervised training method. The machine learning model is trained to take data associated with the plurality of sets of clinical measurements of the infant as input, and output a score indicating a likelihood of an anomaly is present. Responsive to determining that the score is greater than a threshold, the telemedicine system determines that the anomaly is present. In some embodiments, the anomaly indicates that the infant is having a seizure.

In some embodiments, multiple machine learning models are trained to detect different anomalies associated with different clinical conditions. In response to detecting an anomaly, the telehealth system may generate and send an alert. In some embodiments, the alert may be sent to a client device at the monitoring center to notify the experts. The experts may decide whether additional interventions should be performed. In some embodiments, the alert may be sent to a client device at the infant care center to notify a care team that is taking care of the infant.

### Example System Environment

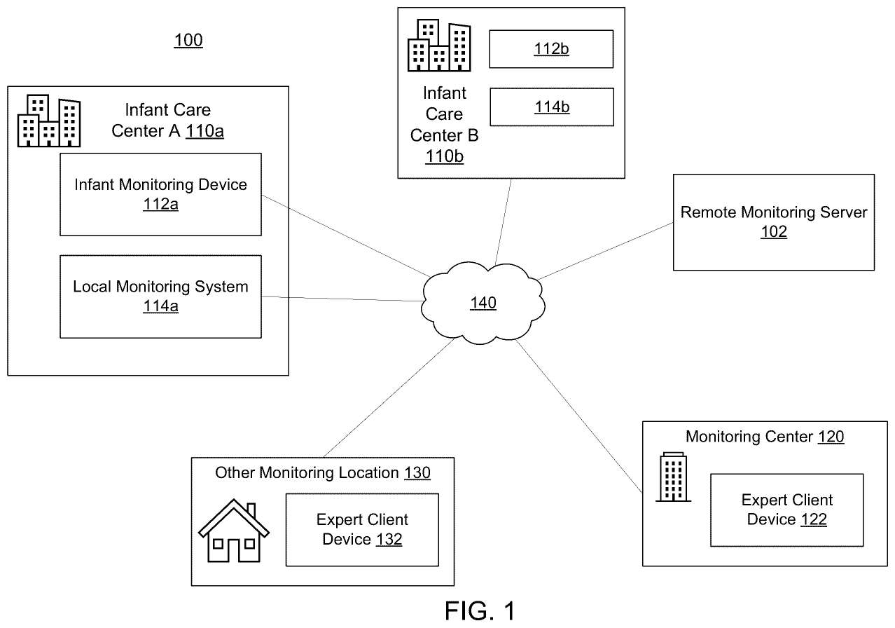

FIG. 1 is a block diagram of a system environment 100 of an infant brain injury monitoring system, in accordance with some embodiments. The system environment 100 shown by FIG. 1 may include remote monitoring server 102, monitoring devices 112, local monitoring systems 114, and expert client devices 122 located at different locations that are remote from the remote monitoring server 102. For example, infant monitoring device 112a and a local monitoring system 114a are located at infant care center A 110a; infant monitoring device 112b and local monitoring system 114a are located at infant care center B 110b. The infant care center A 110a or infant care center B 110b may be a hospital, a home care location, or any other care facility. The local monitoring system 114 may include one or more client devices having a graphical user interface showing data obtained from the local infant monitoring device 112. In some embodiments, the local monitoring system 114 is also configured to transmit the data obtained from the local infant monitoring device 112 to the remote monitoring server 102 via network 140. The remote monitoring server 102 receives data from various locations where infants are being monitored, processes the received data, and causes a graphical user interface (GUI) to be presented at an expert client device 122 and/or 132. The expert client device 122, 132 may be located at any location, such as a monitoring center 120 (where experts reside), or any other monitoring facility 130, which may be a private office or a home of an expert.

The system environment 100 is related to a process for monitoring and managing the health of infants, including preterm infants who are susceptible to brain injury conditions. The process provides real-time monitoring of vital measurements from an infant and presents the measurement data in a graphical user interface, allowing for early detection and notification of potential brain injury conditions. Several risk-prone conditions imply risks for brain injury in the neonatal period. Such conditions include, but are not limited to, hypoxic ischemic encephalopathy (HIE), preterm infants whose gestational ages (GA) are fewer than 28 weeks, infants with cyanotic congenital heart disease (CHD), congenital anomalies (CNS anomalies), seizures, metabolic disorders, severe intraventricular hemorrhage (IVH), critical/unstable or persistent pulmonary hypertension of the newborn (PPHN), central nervous system infections, central nervous system malformations, symptomatic patent ductus arteriosus (PDA), hyperbilirubinemia with total bilirubin greater than 20 or hemolytic process, posthemorrhagic ventricular dilatation (PVD), BRUE (brief, resolved, unexplained event), ALTE (apparent life-threatening event), extracorporeal membrane oxygenation (ECMO).

An infant care center 110 may be a location where infants are physically located and receive care and treatment. An infant care center 110 may include various infant care locations, such as an emergency room, an infant care room, a neonatal intensive care unit, or any other location where infants may receive medical care. Infants may include newborn infants, especially those who are born preterm and/or associated with the abovementioned risk-prone conditions for brain injury. However, in some embodiments, any infants who are receiving medical care or treatment may be monitored under the system environment 100. For those with risk-prone conditions, infants typically need consistent neurocritical care, such as well-trained neonatologists, nurses, and neurologists, who use proper protocols, strategies, and skills, such as brain monitoring protocols, methodologies, neuroprotective strategies, and/or expertise in performing the neuro exam.

The infants at an infant care center 110 may be monitored by one or more infant monitoring devices 112, which generate different sets of clinical measurements. The clinical measurements may include various vital measurements and other medical measurements. The vital measurements of the infant may include, but are not limited to, an infant's heart rate, blood pressure, oxygen saturation levels, temperature, and respiratory rate. Infant monitoring devices 112 may also include common bedside monitors. In some embodiments, the infant monitoring devices 112 may also include end-tidal CO₂, anesthetic agents, cardiac output, etc.

Additional or alternative measurements may include electroencephalography (EEG) or amplitude-integrated electroencephalography (aEEG) monitoring, near-infrared spectroscopy (NIRS) monitoring, and/or therapeutic hypothermia. Near-Infrared Spectroscopy (NIRS) is a non-invasive technology for continuous monitoring of regional tissue hemoglobin oxygen saturation. Amplitude-integrated electroencephalography (aEEG) allows continuous evaluation of cerebral function and seizure screening. Simultaneous use of these techniques may aid in comprehending the physiology of hemodynamic changes and the risk of cerebral injury. As discussed in further detail below, performing brain monitoring with two or more measurements together (e.g., EEG/aEEG) significantly improves seizure detection accuracy, reduces the use of anticonvulsants, and is associated with better neurological outcomes in randomized clinical trials.

An infant monitoring device 112 may also be a sensor for NIRS, which is able to detect earlier biomarkers of brain or somatic injuries. Regardless of systemic oxygenation (SpO2), cerebral or somatic oxygenation may be inadequate. NIRS values have an early correlation with other indicators of systemic poor perfusion (such as high lactate, altered peripheral perfusion, cold extremities, and/or low urine output). Based on the NIRS values, additional interventions, such as ventilator changes, diuretics, change in PGE dose, blood transfusion, and early surgical intervention may be timely performed. In some cases, therapeutic hypothermia is performed, and therapeutic hypothermia is proven to reduce the risk of death or severe neurological impairment.

In some embodiments, infants with HIE may be monitored with amplitude-integrated electroencephalography (aEEG) and continuous electroencephalography (cEEG). In some embodiments, infants with seizures should also be monitored with aEEG and cEEG. In some embodiments, infants with ECMO or pre-ECMO may be monitored with near-infrared spectroscopy (NIRS) and considered to be monitored with aEEG. In some embodiments, infants with grade III or IV IVH or PHVD may be monitored with aEEG. In some embodiments, infants with PPHN may be monitored with NIRS. In some embodiments, infants with GA fewer than 28 weeks may be monitored with aEEG and NIRS. In some embodiments, infants with CNS anomalies, CNS infections, and/or metabolic diseases may be monitored with cEEG and/or aEEG. In some embodiments, infants with cyanotic CHD and/or symptomatic PDA may be monitored with NIRS. In some embodiments, infants with ALTE or BRUE may be monitored with aEEG. In some embodiments, infants with hyperbilirubinemia greater than 20 or hemolytic process may be monitored with NIRS and be considered to be monitored with aEEG.

The remote monitoring server 102 may be a server that is located geographically remote from an infant care location. In some embodiments, the remote monitoring server 102 may take the form of a Cloud server. In other embodiments, while the remote monitoring server 102 may be located remotely from an infant care location, but may still be located within an infant care center 110. For example, an infant care center 110 may provide care to multiple infants in various infant care locations. The remote monitoring server 102 may be a server located at the infant care center 110. The real-time measurement data collected at various infant care locations may be transmitted to the server for further analysis, synchronization and/or aggregation.

In some embodiments, a monitoring center 120 may be a centralized location that allows medical professionals to monitor one or more infants' medical conditions. The monitoring center 120 may also be referred to as a monitoring facility. The medical professionals may be experts who are trained to monitor and detect signals of potential risks of brain injury. In some embodiments, a monitoring center 120 may be located geographically remote from an infant care location or even the infant care center 110. For example, in some embodiments, various infant care centers 110 may transmit real-time measurement data of various infants to an offsite monitoring server 102. The real-time data, for each infant, are aggregated and displayed in a time-synchronized manner as multiple time series on a monitor located at the monitoring center 120. The experts at the monitoring center 120 may monitor those infants remotely. In some embodiments, the experts may rely on their expertise, experience, and machine-generated signals and warnings to determine whether an infant is at risk of brain injury. In some embodiments, a monitoring center 120 may be located within an infant care center 110. For example, instead of sending data to an offsite monitoring center, the monitoring center 120 may be a nurse room, a doctor room, or another monitoring location where one or more medical professionals on-site may monitor the conditions of the infants at an infant care center 110. The physical arrangements of the infant care center 110, the remote monitoring server 102, and the monitoring center 120 may vary in different embodiments.

The real-time data associated with the plurality of sets of clinical measurements of the infant may be stored as a plurality of time series data in a data store associated with the remote monitoring server 102. The stored data can then be used to cause a graphical user interface to present the plurality of time series data in a time-synchronized manner on a monitor located at a monitoring facility. For example, the monitor may be a monitor of an expert client device 122.

The graphical user interface presents the plurality of time series data in a time-synchronized manner on a monitor located at the monitoring center 120. The time series data is displayed in real-time as the clinical measurements are taken at the infant care location, allowing the medical personnel at the monitoring facility to track the health of the infant in real-time. In some embodiments, the graphical user interface may also display other relevant information, such as alerts for critical or abnormal readings, or trending data for specific vital measurements over time.

In some embodiments, the graphical user interface can present vital measurement data in a variety of ways, such as graphs, charts, or numerical displays. For example, the graphical user interface can display a real-time graph of the infant's heart rate, oxygen level, and blood pressure in a single graph over time, allowing for easy monitoring of changes in the infant's vital signs. The graphical user interface can also present a numerical display of the infant's oxygen saturation levels and respiratory rate, allowing for easy monitoring of these vital signs.

The clinical measurements are generated in real-time at the infant care location and are displayed at the monitoring center 120 in a time-synchronized manner in real-time. There may be a latency between the measurement and display at the monitoring center 120 due to network transmission, data analysis, and other computing delays. The data are displayed at the monitoring center 120 in a clinically acceptable time delay. In some embodiments, the delay is within 100 ms. In some embodiments, the delay is within 200 ms. In some embodiments, the delay is within 300 ms. In some embodiments, the delay is within 400 ms. In some embodiments, the delay is within 500 ms. In some embodiments, the delay is within 1s. In some embodiments, the delay is within 2s. In some embodiments, the delay is within 3s. In some embodiments, the delay is within 4s. In some embodiments, the delay is within 5s. In some embodiments, the delay is within 10s. In some embodiments, the delay is beyond 10s as long as it is determined that the delay is clinically acceptable.

In some embodiments, the remote monitoring server 102 may receive a communication from the monitoring center 120 indicating that the infant is at risk of a potential brain injury condition. Upon receipt of this communication, the remote monitoring server 102 may provide a notification to the infant care location regarding the potential brain injury condition. This notification can be used to prompt appropriate action to be taken at the infant care location to prevent or manage the potential brain injury condition. For example, the notification can alert medical personnel at the infant care location to the potential risk of a seizure and prompt them to take measures to prevent the seizure from occurring, such as administering medication or adjusting the infant's position.

In some embodiments, the data store associated with the remote monitoring server 102 can be configured to store the plurality of time series data for a specified period of time, allowing for historical data to be reviewed and analyzed. This historical data can be useful in monitoring trends in the infant's health, identifying potential health issues, and providing a record of the infant's health status. For example, if the infant has a history of seizures, the historical data can be used to identify patterns or triggers that may be associated with the seizures, allowing for proactive measures to be taken to prevent the seizures from occurring.

In some embodiments, the monitoring center 120 can be staffed by neonatologists, pediatricians, nurses, or other medical personnel trained to monitor the health of infants. In some cases, the monitoring facility may be located in a hospital, while in other embodiments, it may be located in a separate location specifically designed for monitoring infants.

The real-time data associated with the plurality of sets of clinical measurements are stored in a data store associated with the remote server. This data store can be a database or other type of storage system capable of efficiently storing and retrieving large amounts of time-series data. The data store can be securely protected to ensure the confidentiality and privacy of the infant's health information.

If the monitoring center 120 determines that the infant is at risk of a potential brain injury condition, such as a seizure, they can communicate this information to the remote server. Upon receiving this communication, the remote server can provide a notification to the infant care location regarding the potential brain injury condition. The notification can be sent to the infant care location via email, text message, or other means of communication, and can include relevant information about the potential brain injury condition, as well as any recommended actions or protocols.

### Example Server Components

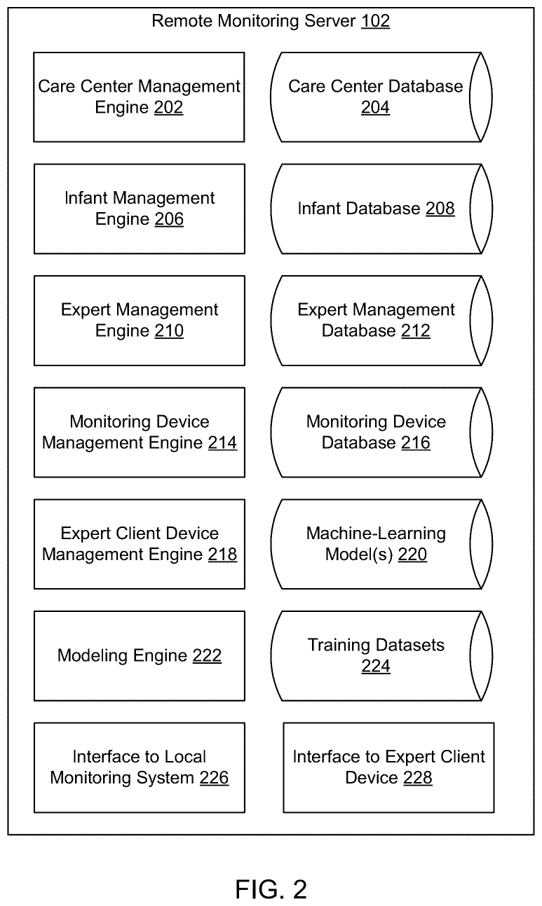

FIG. 2 is a block diagram illustrating an example architecture of the remote monitoring server 102. The remote monitoring server 102 includes a care center management engine 202 and a care center database 204. The care center database 204 stores data associated with each infant care center, such as infant care center 110a, 110b. The care center management engine 202 allows an administrator of the server 102 to manage the care center database 204, such as adding new infant care centers or modifying existing infant care centers. The remote monitoring server 102 also includes infant management engine 206 and patient database 208. The infant database 208 stores data associated with each infant that is being monitored. The infant management engine 206 allows an administrator of the server 102 or a particular care center to manage data associated with infants who are being monitored at the particular care center, such as adding a new infant into the infant database 208, and/or modifying or deleting an existing infant from the infant database 208.

In some embodiments, the remote monitoring server 102 also includes an expert management engine 210 and an expert management database 212. The expert management database 212 stores data associated with each expert that has the expertise and is allowed to review the infant monitoring data. The expert management engine 210 allows an administrator of the server 102 to manage data associated with the experts, such as adding a new expert into the expert management database 212, and/or modifying or deleting an existing expert from the expert management database 212.

In some embodiments, the remote monitoring server 102 also includes a monitoring device management engine 214 and a monitoring device database 216. The monitoring device database 216 stores data associated with each device that is used to monitor an infant. The monitoring device management engine 214 allows an administrator of the remote monitoring server 102 or a particular infant care center to manage data associated with monitoring devices, such as adding a new device, removing a device, linking a device to a particular infant, etc.

In some embodiments, the remote monitoring server 102 also includes an expert client device management engine 218 to register each expert client device, and link the expert client device with a particular expert or a particular monitoring center.

In some embodiments, the remote monitoring server 102 also includes machine learning models 220 trained to detect anomalies associated with clinical conditions. In some embodiments, the remote monitoring server 102 also includes a modeling engine 222 and training datasets 224. The modeling engine 222 is configured to use the training datasets 224 to train the machine learning models 220. The machine learning models 220 are trained to receive data associated with clinical measurements of an infant as input, to output a prediction of whether the infant experiences an anomaly associated with a clinical condition.

In some embodiments, the remote monitoring server 102 also includes an interface 226 for communicating with local monitoring systems 114a, 114b. In some embodiments, the remote monitoring server 102 also includes an interface 228 for communicating with expert client devices 122, 132. For example, the interface 226 is configured to receive infant monitoring data from the local monitoring systems 114a, 114b. The interface 228 is configured to transmit the received infant monitoring data to the expert client devices 122 and/or 132, and/or receive input from the expert client devices 122, 132. The input received from the expert client device 122, 132 may indicate whether an expert believes that a particular infant needs a treatment intervention. After receiving such an input, the interface 226 may then send an alert to the local monitoring system 114a, 114b.

### Local Monitoring System Overview

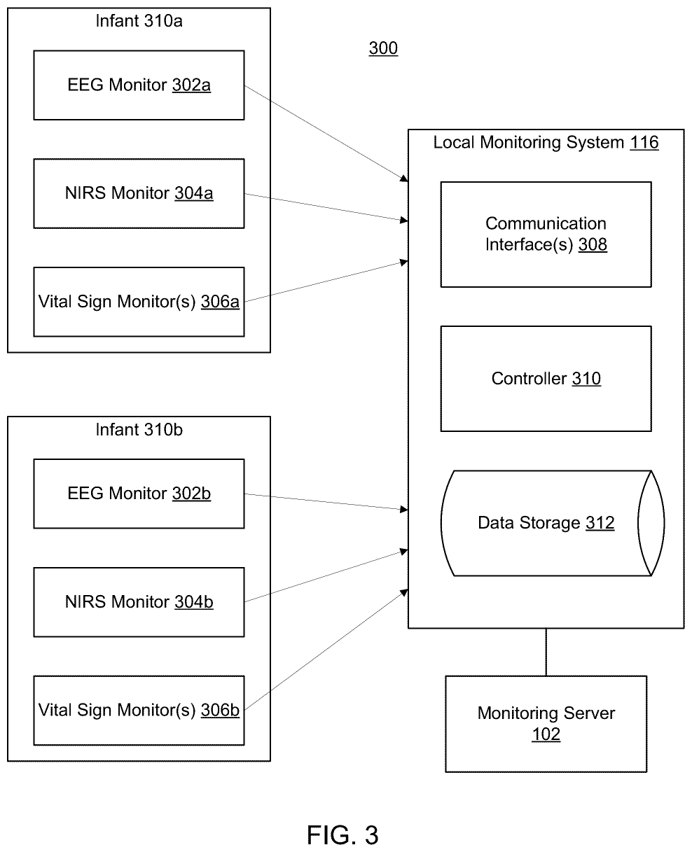

FIG. 3 is a block diagram illustrating an environment 300 in which a local monitoring system 116 resides. The environment 300 may be an infant care center 110a or 110b. The environment 300 include one or more infants, e.g., infants 310a, 310b. For each infant 310a, 310b, one or more monitoring devices are used to perform clinical measurements. Such monitoring devices may include (but are not limited to) an EEG monitor 302, an NIRS monitor 304, and/or vital sign monitor(s) 306. Each of the monitors 302, 304, 306 is connected to the local monitoring system 116.

The local monitoring system 116 may be a generic computer system having specialized software installed thereon or a specialized computer device having firmware or software installed thereon. The local monitoring system 116 includes one or more communication interfaces 308, a controller 310, and a data storage 312 configured to receive data from various monitoring devices 302, 304, 306, and transmit the received data to the remote monitoring server 102. In some embodiments, the local monitoring system 116 may also be configured to receive alert from the remote monitoring server 102 responsive to detecting an anomaly of one of the infant 310a, 310b.

### Example Graphical User Interfaces (GUIs)

Once the remote monitoring server 102 receives the infant monitoring data associated with multiple clinical measurements of the infant, the remote monitoring server 102 aggregates the data over a period of time, and generates a GUI displaying the aggregated data associated with the multiple clinical measurements simultaneously. The GUI is then caused to be presented on an expert client device 122, 132 at a monitoring facility 120, 130.

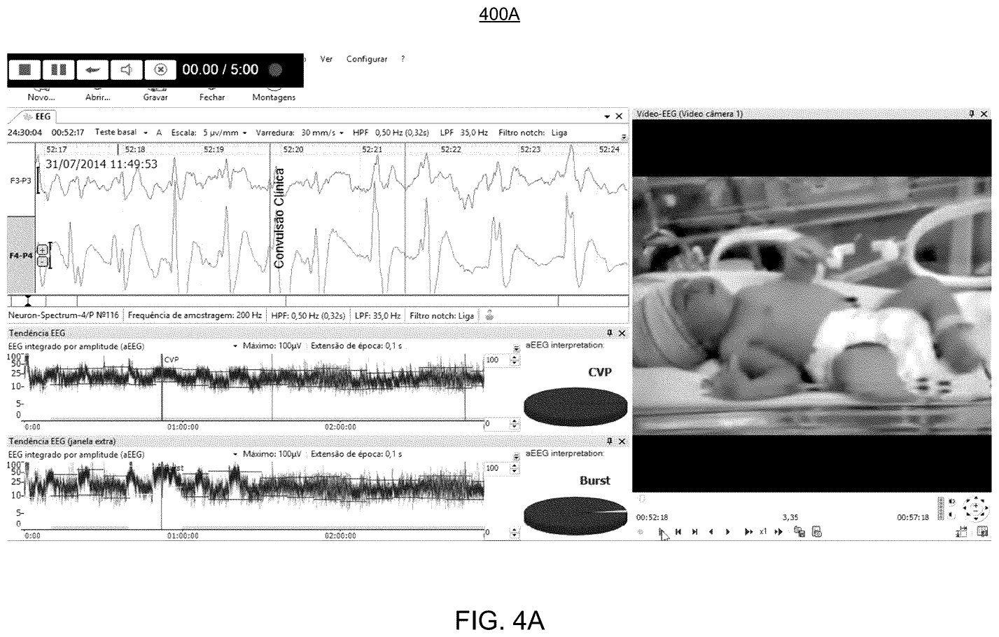

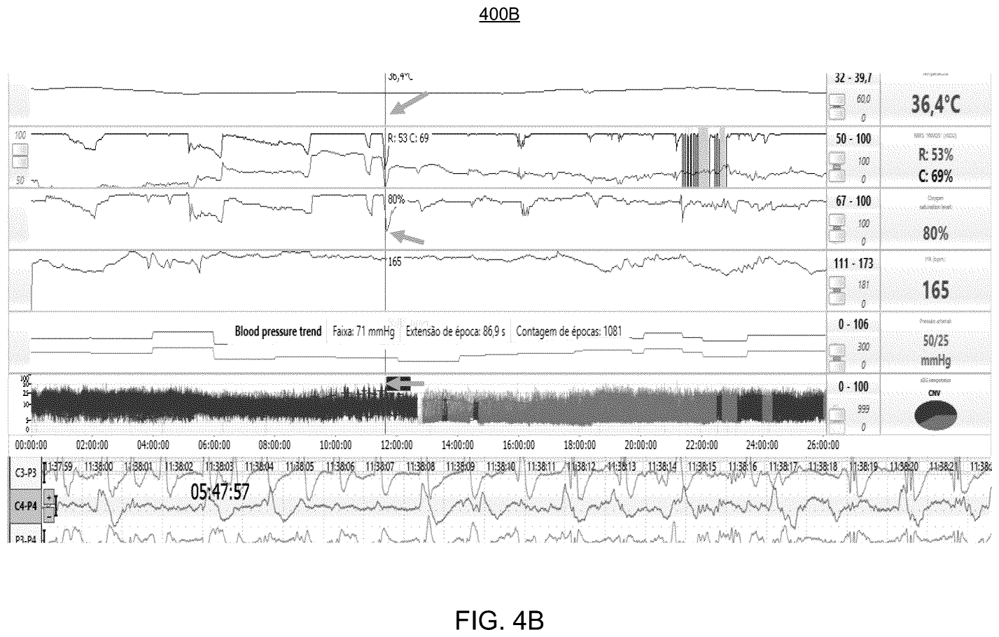

FIGS. 4A and 4B illustrate example GUIs 400A, 400B displaying aggregated monitoring data of an infant. GUI 400A of FIG. 4A shows data associated with an infant generated based on video aEEG. Based on the data shown in GUI 400A, an expert can diagnose that the infant is likely experiencing a focal clonic seizure. GUI 400B of FIG. 4B shows data associated with an infant generate based on aEEG/EEG, NIRS, and vital signs. Based on the data shown in GUI 400B, an expert can diagnose that the infant is experiencing frequent desaturations associated with seizures. In the case shown in GUI 400A or 500B, the expert can prescribe a proper treatment intervention responsive to the diagnosis, mitigating potential brain injury.

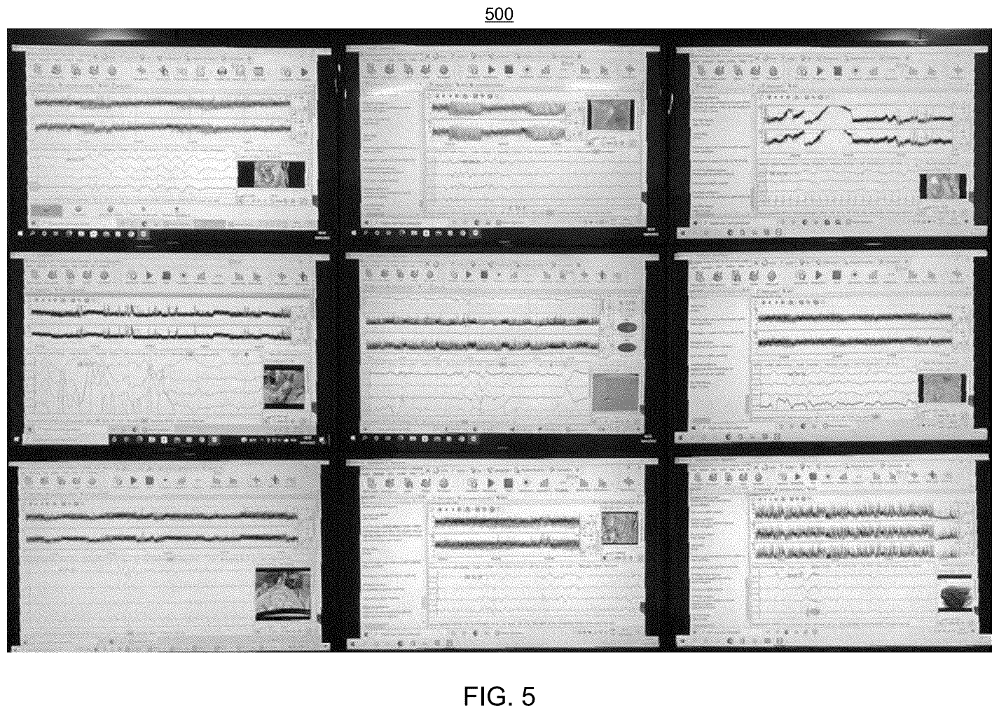

FIG. 5 illustrates an example GUI 500 displaying monitoring data of multiple infants simultaneously. GUI 500 may be displayed at a single large screen or an array of screens at a monitoring center 120.

### Multi-Modality Monitoring Software

In some embodiments, multi-modality monitoring software (hereinafter also referred to as “the software”) is implemented at the remote monitoring server 102. Alternatively, partial functions of the software are distributed at the local monitoring system 114, infant monitoring device 112, and/or expert client device 122, 132. The software is designed to allow integration from multiple different types of monitors (e.g., aEEG/EEG, NIRS, and vital signs) and data streaming in real-time to remote monitoring server 102 allowing remote access of this information in a time-synced fashion.

The software performs multiparametric monitoring of patients (i.e., infants) in ICU, allowing access to the different vital signs and brain monitoring information (aEEG/EEG, ECG, SPO2, blood pressure, heart rate, capnography, and NIRS). The analysis of the correlations between the findings allows a greater understanding of physiology and may improve diagnostic accuracy. The software creates a platform for the centralization of monitoring data, creating mechanisms that allow interpreting and correlating data from each patient, generating alarms in case of critical situations. Further, the software is the basis for integration to allow the use of artificial intelligence algorithms created from the collected data.

In particular, the software stores the continuous monitoring data from ICU patients, and visualizes parameters in a time-synchronized manner by evaluating trends at different time frames. In some embodiments, the time frame may be edited or modified by users, such as an expert, an administrator of a local monitoring system 114, or an administrator of the remote monitoring server 102. In some embodiments, the software is configured to add average lines to the visualization to show monitoring trends. In some embodiments, the software generates a report periodically for each patient, such as every 24 hours. The report evaluates the patient's evolution over the period (e.g., hours or days) of monitoring. In some embodiments, the software replaces current evaluation of vital signs parameters within the infant care center, such as an ICU (which may occur by intermittent annotations, such as every 2 hours) by a continuous and much more reliable evaluation. In some embodiments, the software allows comparative analysis and correlation between different monitoring parameters continuously. In some embodiments, a user of the platform can set up individual alarm parameters according to each monitored patient profile (e.g., term, preterm, with congenital heart disease, etc.). In some embodiments, the software creates a risk score, evaluating subtle abnormalities and making earlier diagnoses of brain and organic injury.

In some embodiments, the software generates user-friendly GUIs that allow easy review of patients' multiple monitored parameters inside the ICU. In some embodiments, the GUIs enable interactions between remote experts and bedside teams, and integrations between bedside monitoring and monitoring at monitoring center. In some embodiments, the software stores and classifies information according to a studied population, allowing the creation of databases of different populations for machine learning.

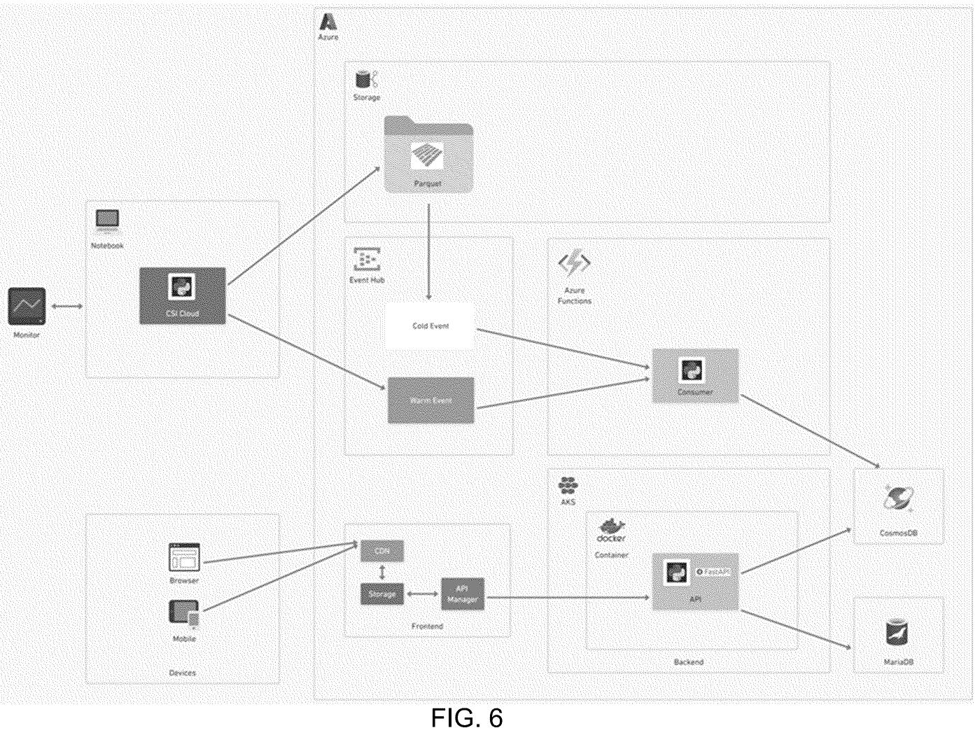

FIG. 6 illustrates an example architecture of the software, enabling the collection of monitoring data and providing a responsive web interface. The software may be hosted at a cloud service provider, such as (but not limited to Microsoft Azure®). The software includes an active directory, a parquet, an event hub, azure functions, CDN, storage account, an API manager, a webApp, an AKS Kubernets, a CosmosDB, and a MariaDB. The active directory serves as a cloud domain controller. The parquet is a column file format that provides optimizations to speed up queries. The event hub performs data collection from peripherals (e.g. notebooks). The Azure functions perform data transport process. CDN performs high-bandwidth content delivery. The storage account is a cloud storage unit. The API manager is an API services manager. The WebApp hosts service and performs interface provision. The AKS Kubernetes is a storage of API-type applications. The CosmosDB is a non-relational database. The MariaDB is a patient/infant data storage.

The data from bedside monitors are exported to a central hub (notebook/computer) at the bedside in the neonatal ICU, and it is sent to the cloud service, stored on a blob storage volume, transformed and processed into Parquet format to optimize and speed up queries. After that, the Event hub processes queue requests and starts threads on the main process that occurs on Azure functions. After that, the transformed data is stored on CosmosDB and published on a portal of the platform. CDN architecture provides high bandwidth for data storage, reducing outbound costs.

In embodiments, to guarantee high availability and scalability, the application uses AKS Kubernetes farm that consumes data from Storage Account and MariaDB. All the environment runs under Azure Active Directory access control, and the entire API layer is authenticated via dynamic token, and its IP address is assigned to Azure NSG (Network Security Group).

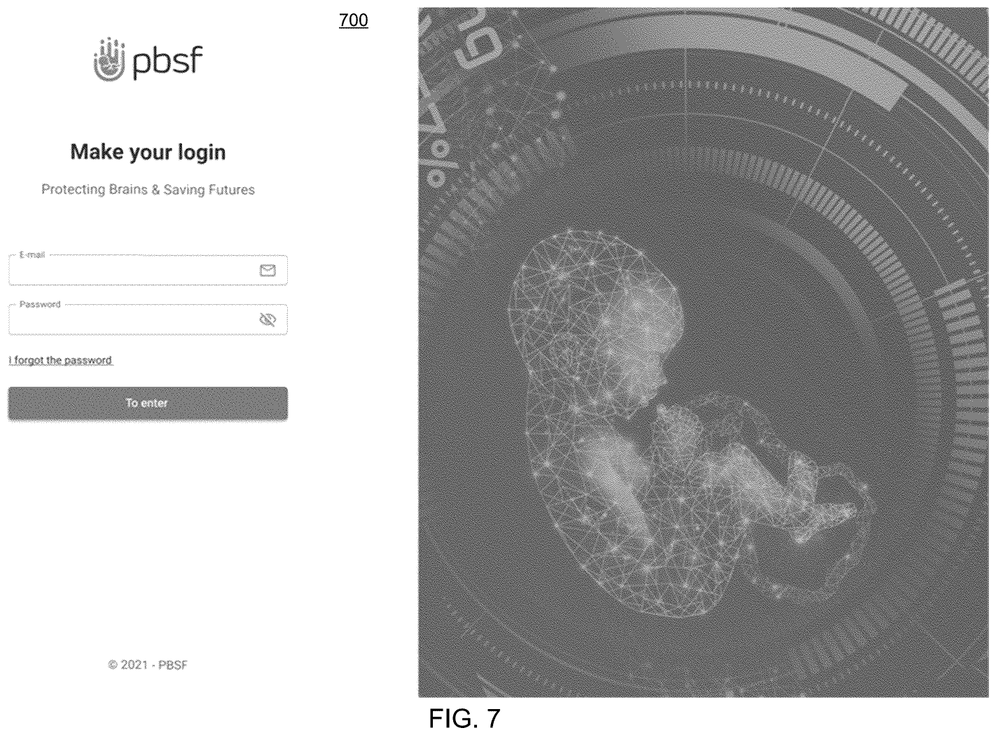

The software allows authorized users to login to the platform to review the patient/infant monitoring data. The users may be healthcare providing teams at the infant care center, experts at monitoring facility, and/or family members of the patients. FIG. 7 illustrates an example GUI 700 allowing users to login.

In some embodiments, after a new user is created, a password is automatically generated and sent to an email provided by the new user, e.g., a random string is generated as the password. After the user logs in, the user can change the password to a more secure or preferred password. In some embodiments, administrator users can only perform user registration. In some embodiments, password creation is required to follow certain patterns, such as the password must have a minimum of 8 and a maximum of 10 characters, the password must be at least one letter, the password must have at least one lowercase letter, the password must have at least one special character, the password must have at least one number, etc. Further, the platform may also set password entering requirement. For example, after inserting the wrong password 3 times, the user will be blocked until it is recovered or released by forgetting my password function. In embodiments, passwords must be encrypted in cryptographic form or equivalent.

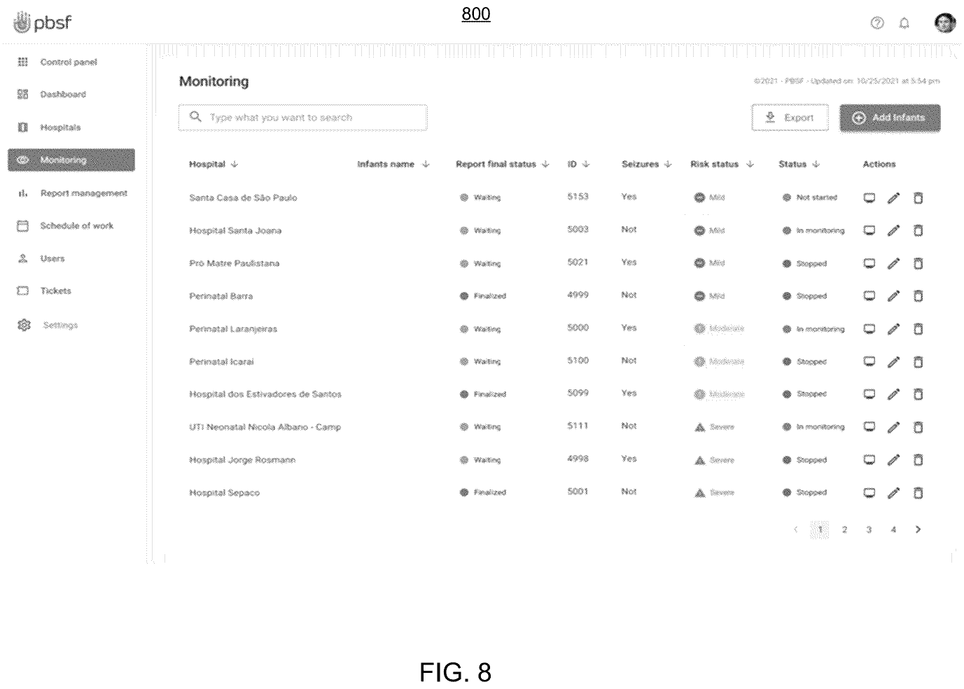

In some embodiments, the software displays a list of monitored patients to an authorized user after the authorized user logs in. FIG. 8 illustrates an example GUI 800 displaying a list of monitored patients. In some embodiments, the software provides an option for the user to export the list, create a new patient, and/or analyze data of each patient through various action buttons. All information related to the patients' data are stored and handled according to the security requirement of the infant care center, such as hospitals. Personal data (e.g., names or identifiers) may or may not be required to be stored by the software.

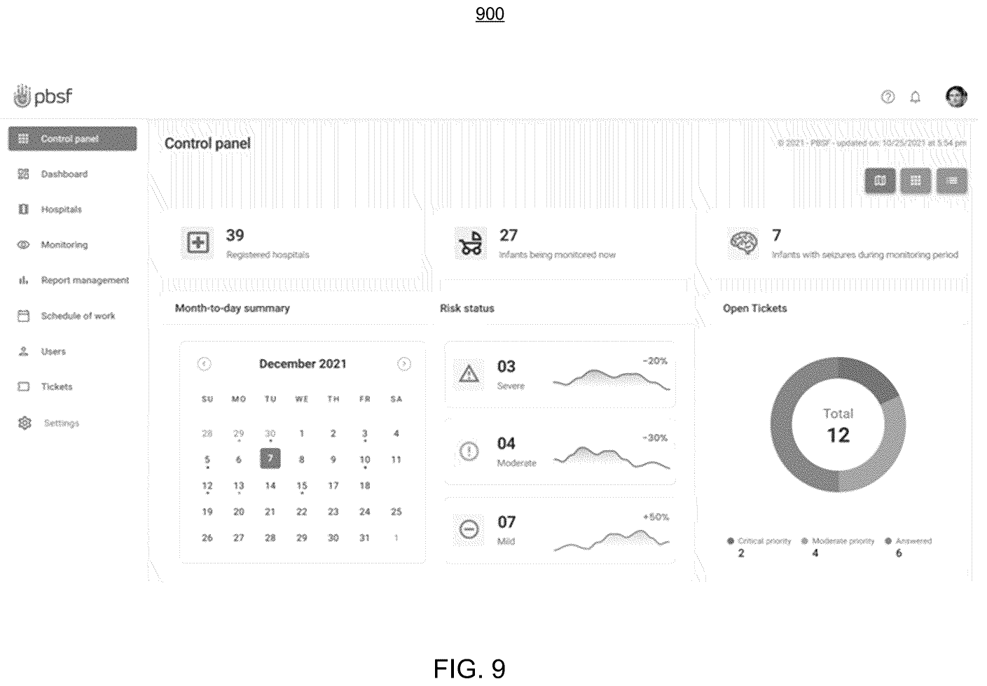

In some embodiments, the software provides a control panel that allows an overview of monitored information, including registered hospitals, a number of monitored infants, infants with seizures, and open tickets (e.g., communications between bedside and remote team). FIG. 9 illustrates an example GUI 900 that shows the control panel.

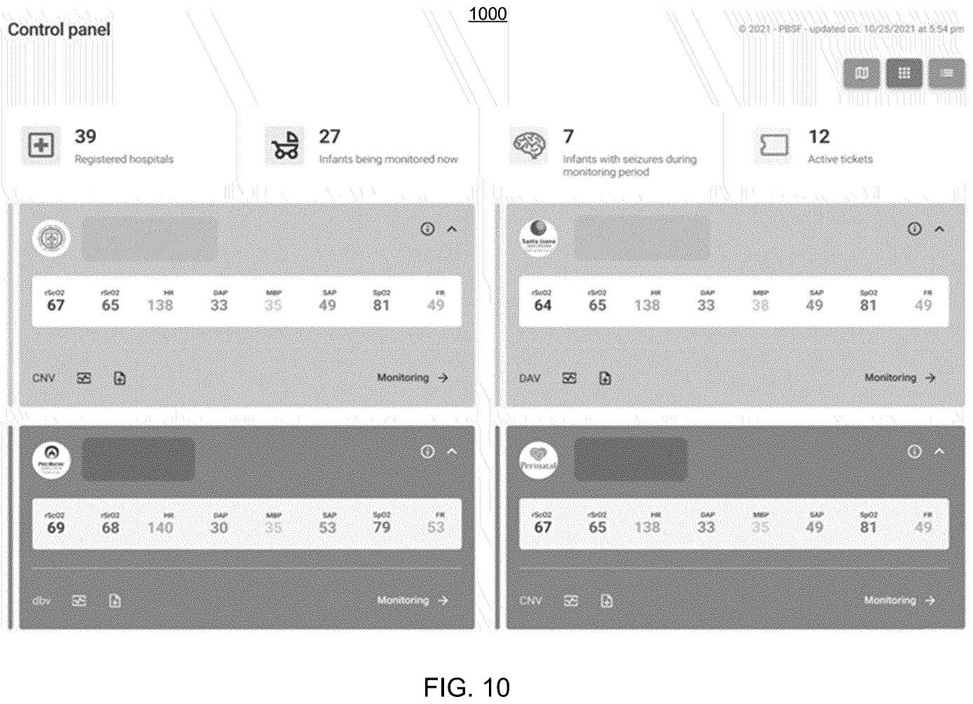

In some embodiments, the software provides different views for the control panel, such as a card view that summarize monitoring data of all infants in different cards. FIG. 10 is an example GUI 1000 showing an example card view of the control panel. As shown in GUI 1000, monitoring data of all infants are summarized in the panel and updated every 30 seconds. Infants are classified to a risk status color coded with green, yellow, and red based on analyzed parameters (e.g., hart rate, SpO2, NIRS values). For example, lower risk infants are color coded as green, high risk infants are color coded as red, and medium risk infants are color coded as yellow.

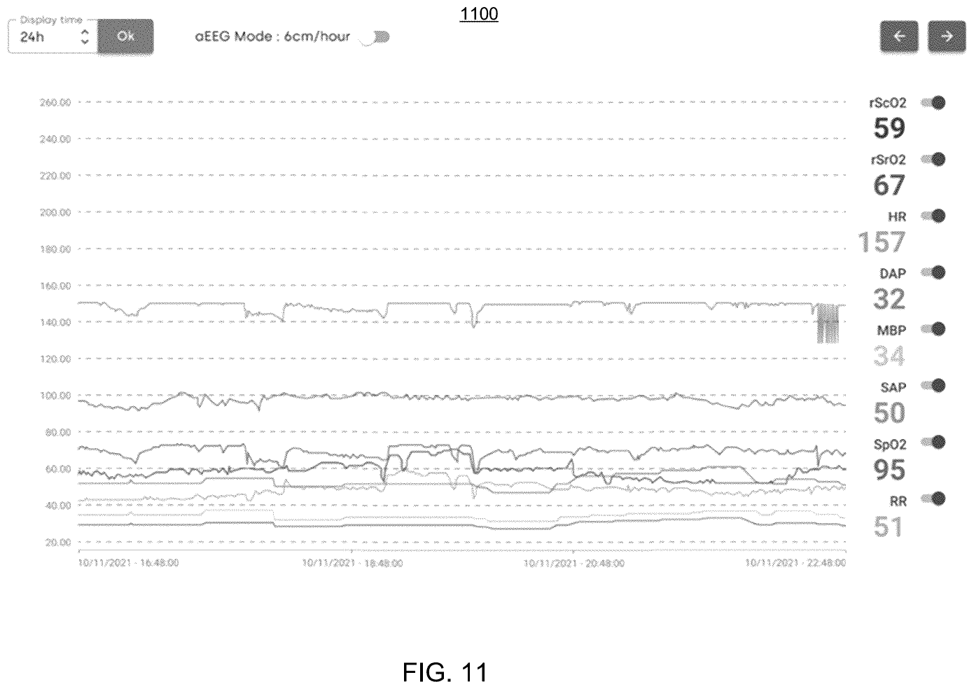

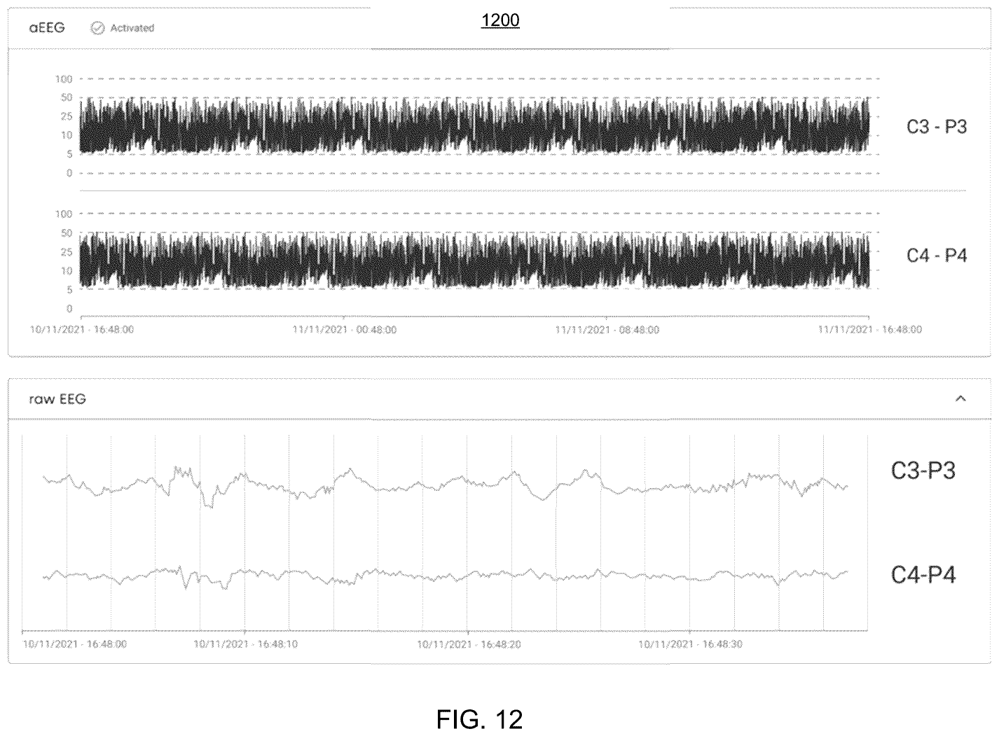

In some embodiments, information from NIRS monitoring (e.g., rScO2 and/or rSrO2), vital signs (heart rate (HR), SpO2, blood pressure, resp rate) are time-synced nd displayed on GUI. The display time can be adjusted, such as 6 to 24 hours of monitoring information, with the possibility of navigation along the tracing. FIG. 11 is an example GUI 1100 displaying data associated with NIRs and vital signs. The GUI 1100 may present a plurality of time series data in a time-synchronized manner on a monitor located at a monitoring center 120. The plurality of time series data may be displayed in real-time as the plurality of sets of clinical measurements are measured at the infant care location 110. Additional GUI examples related to how data are synchronized and presented in an aggregated manner are shown in FIG. 12 through FIG. 17.

In some embodiments, two-channel aEEG and raw EEG readings are displayed on the GUI. FIG. 12 is an example GUI 1200 displaying data associated with aEEG and raw EEG.

In some embodiments, the software uses a multivariate anomaly detector algorithm to automatically detect seizures of patients, e.g., infants in NICUs. A significant barrier to the practice of neuroprotective critical care in NICU is the lack of expertise in reporting neonatal EEG. Current bedside EEG monitors are sophisticated devices that offer the ability to record multiple channels of EEG continuously together with other physiological signals and video recording the infants' movement. Such devices allow the continuous display of aEEG and other quantitative trends, and are easy to set up and maintain. However, very few clinicians have the knowledge to interpret the plethora of information generated by such devices. Without such knowledge, the devices are underutilized, or the output may be misinterpreted at the bedside.

The software described herein use advanced data acquisition techniques and machine learning technology to perform automated seizure detection for newborn population. The amount of time series data generated by equipment accumulates quickly. The software is configured to build an unsupervised multivariate time-series anomaly detection model configured to detect anomalies on multiple signals. In general, multi-univariate time series from a same device form a multivariate time series. It is advantageous to directly analyze time series at the entity level using multivariate time series rather than at any individual univariate time series. In practice, the health status of an entity cannot be reflected by any individual signal.

A neonatal intensive care unit (NICU) is a place where life altering decisions are constantly made in order to provide care to ill patients such as infants below 1,000 g. neonatologists compile data from a variety of sources to build a picture of a newborn's health condition in order to ensure they receive the best medical care available. Highly trained experts use their judgment in conjunction with a continuous stream of patient data to ensure that the best potential outcome is achieved for the largest number of infants. The use of machine learning and/or artificial intelligence enhances the decision making process and lead to better outcomes.

Notably, high frequency EEG (200 Hz)/EEG (150 Hz) signals from medical equipment are hard to analyze manually. There is no intuitive way to capture abnormal events through those signals. Automatically monitoring those signals by machine learning can help physicians to diagnose and treat patients in time. Further, multi-modality patient data can be collected when separate monitoring devices are linked to a centrally placed server through a network. The technique retains the benefit of synchronized timing and adds the use of primarily automatic capture, requiring minimum human input to start and stop the recording. The multi-modality approach combines brain monitoring techniques with other vital signs and clinical information to study systemic and cerebral hemodynamics and electrographic findings early after birth, allowing a better understanding of the critically ill infant physiology. Further, the machine learning based anomaly detection is better than rule-based anomaly detection when there are multiple signals to be monitored, and abnormal patterns are hard to be described by pre-existing rules.

In healthcare, most of the data are unlabeled or with sparce labels. Thus, it is advantageous to use unsupervised learning. On the feature engineering side, effective representation of time series can be generic for detecting anomalies in different diseases compared with features defined manually. Further, compared with image and video signals, time-series signals can be obtained much easier. Analyzing time-series signals to capture anomalies is more convenient for diagnosing.

The neonatal period, especially the first week after birth, is the most susceptible time of human life for seizure development. Neonatal seizures occur in 1 to 3 per 1,000 live births and are commonly related to acute brain injury, associated with increased mortality and impairment. Neonatal seizures may constitute a neurological emergency, making monitoring and seizure accurate identification critical components of newborn intensive care management. However, seizure detection in the new born is challenging. About 80% of neonatal seizures are not correlated with any clinical signs. Moreover, clinically suspected episodes may not show corresponding electrographic evidence of seizures and may be wrongly diagnosed. Previous studies have shown that treating subclinical seizures is associated with reduced seizure burden and better neurological outcomes. On the other hand, seizure overdiagnosis and treatment are potentially harmful to the developing brain as well.

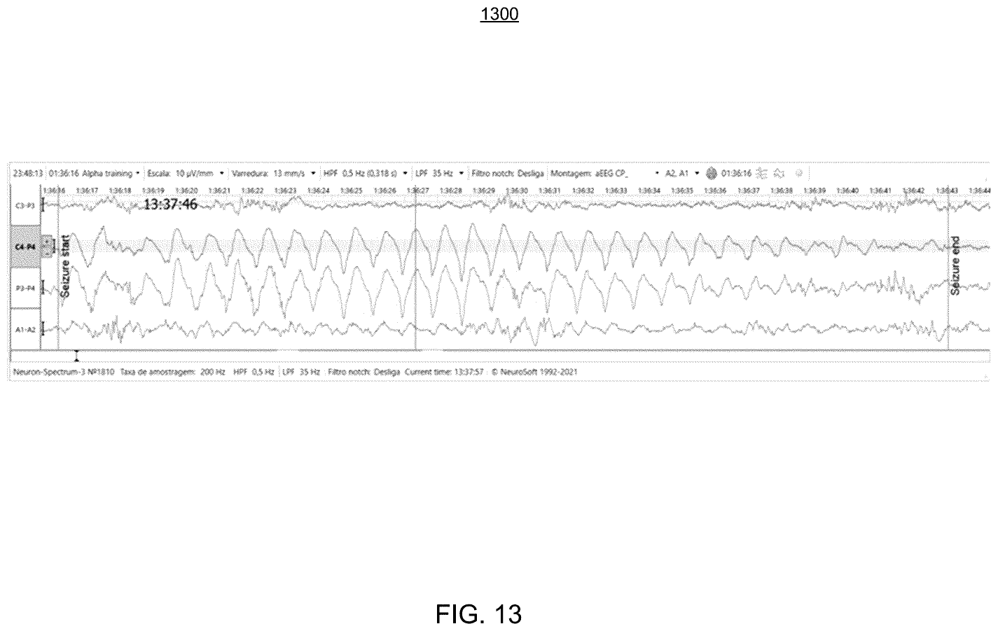

Conventional continuous long-term EEG (cEEG) is considered the gold standard for neonatal seizure detection. However, there are barriers to implementing this technology since cEEG requires skilled technologists, and experienced neurologist interpretation and may not be readily available in many centers, especially regarding continuous bedside monitoring. Continuous monitoring using a one- to two-channel amplitude-integrated EEG (aEEG) with concurrent unprocessed EEG has become an interesting option to be used as a bedside tool for monitoring neonates at high risk. Several studies and reviews have assessed the accuracy of aEEG for seizure detection. Seizure activity is characterized in aEEG by a sudden change in background activity as an abrupt rise in minimum and maximum aEEG amplitude correlated with a stereotypical, repeating form such as spikes or sharp waves, often with high amplitude in raw EEG, with a total duration of at least ten seconds. FIG. 13 is an example GUI 1300 illustrating a neonatal seizure moment.

A machine learning model is trained to detect neonatal seizure moments. A seizure moment is defined as a segment of values. This segment has a different pattern from normal segments. Usually, a real seizure could last more than 10 seconds, and it could be inaccurate if a single timestamp value is used to judge seizure. Thus, in the machine learning model, teim-series representation is learned with contrastive learning, and a classifier is built with sparse label.

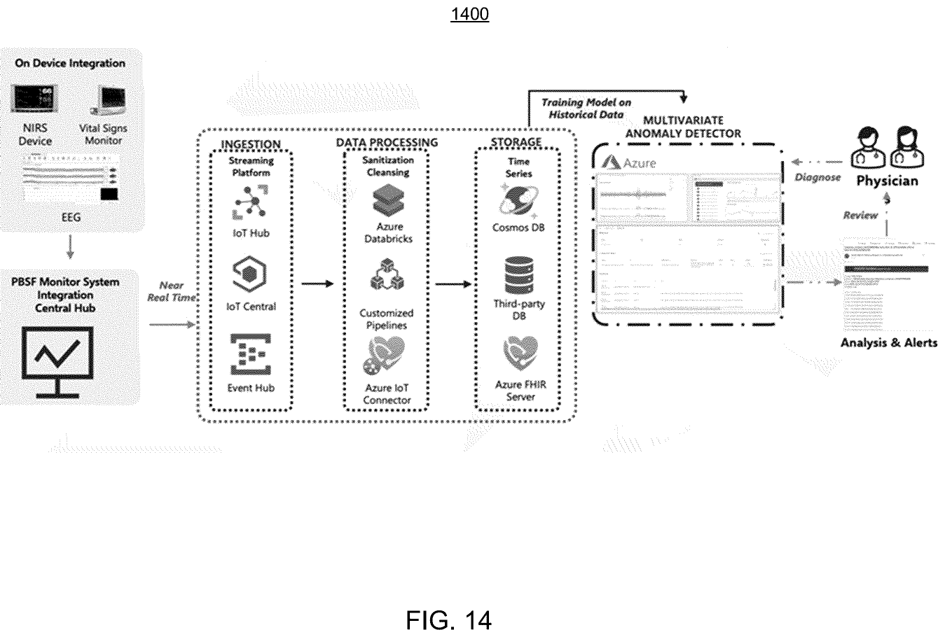

FIG. 14 illustrates an example learning framework 1400 for training the machine learning model for detecting neonatal seizure moments. A cloud service receives data from multiple devices for training. Multiple types of sources are used for collecting signals, including NIRS device, vital signs device, and EEG device. The collected signals include (but are not limited to) EEG, heart rate, temperature, pulse oximetry, and regional tissue oxygen saturation levels. The collected signals are sent to the cloud service and stored for training. Users can manage models on the cloud and export the model for local inference. The trained model is configured to detect and collect signals in local box, and generate alert once anomalies are detected.

Further, the software can use non-invasive monitoring data to determine a probability of presence of cerebral autoregulation in critically ill infants. Germinal matrix-intraventricular hemorrhage (GMH-IVH) is a common complication of premature birth, with a global estimated incidence of 35% among very preterm infants, and represents a possible risk factor for adverse neurodevelopmental outcome. Nearly 80-90% of GMH-IVH occur during the first 72 h of life, which are characterized by the progressive hemodynamic transition from a fetal to a neonatal circulation.

The intrinsic fragility of the germinal matrix vasculature and cerebral blood flow disturbances are major contributors to the multifactorial etiology of GMH-IVH. Among other enteropathogenic mechanisms, the role for an early impairment of cerebral autoregulation (CA), which leads to blood pressure-passive brain perfusion, has been reported. A better understanding of the early hemodynamic changes associated with GMH-IVH development would be helpful to reduce or prevent this complication.

Cerebral autoregulation (CA) is considered one of the most important physiological mechanisms to maintain a constant cerebral blood flow (CBF) and prevent hypo or hyper-perfusion, despite changes in arterial blood pressure (ABP).

Near-infrared spectroscopy (NIRS) is a non-invasive device that measures regional cerebral oxygen saturation (rSO2), which can be used as a surrogate of CBF to measure CA. rSO2 is a measure of the balance between cerebral oxygen delivery (to which CBF is a major contributor) and utilization (associated with the cerebral metabolic rate for oxygen). The cerebral oximetry index (COx) is calculated by the moving correlation between the slow waves of rSO2 and mean arterial pressure (MAP).

In some embodiments, the software calculates and analyze the Cox and provides surrogate information about cerebral autoregulation in near real time. This information is then used to guide clinical interventions (e.g., avoidance of inotropes or fluid bolus) and decreases the chance of brain injury in a susceptible population.

In some embodiments, the software analyzes the cerebral/renal oximetry coherence index between rScO2×rSrO2 (CROx) to create two coherence/correlation index, including (a) IBP (MAP)×rScO2; (Cox), (b) rScO2×rSrO2 (CROx), and/or (c) rScO2×rSrO2×heart-rate×SpO2 (second validation).

There should be a good correlation between Cox and CROx. CROx can work as a surrogate non-invasive measurement of cerebral autoregulation. A high coherence index between rScO2 and rSrO2 can be associated with impaired cerebral autoregulation in neonates. A low coherence index between rScO2 and rSrO2 can be associated with intact cerebral autoregulation in neonates.

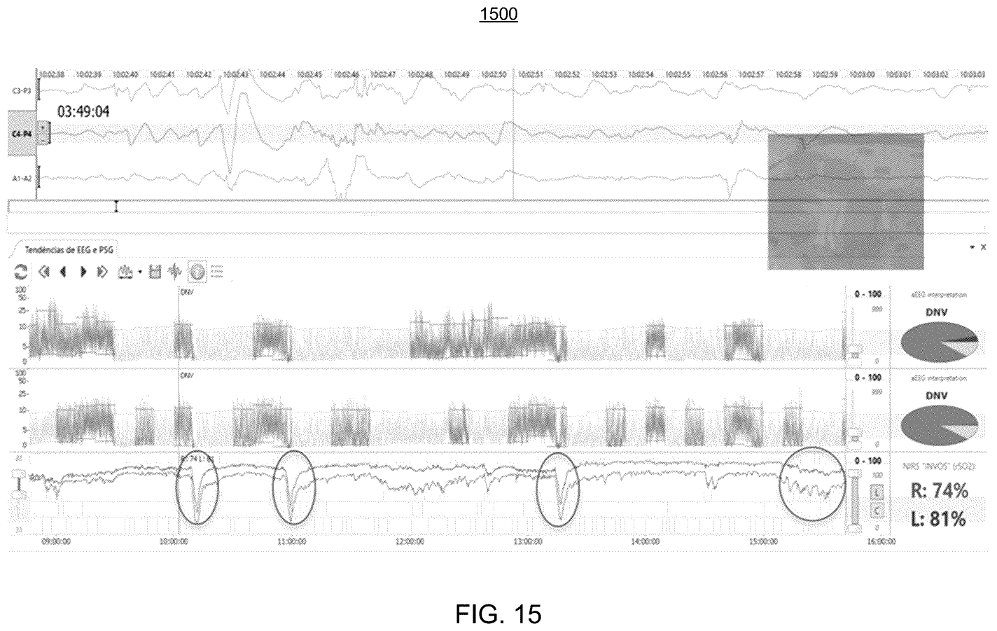

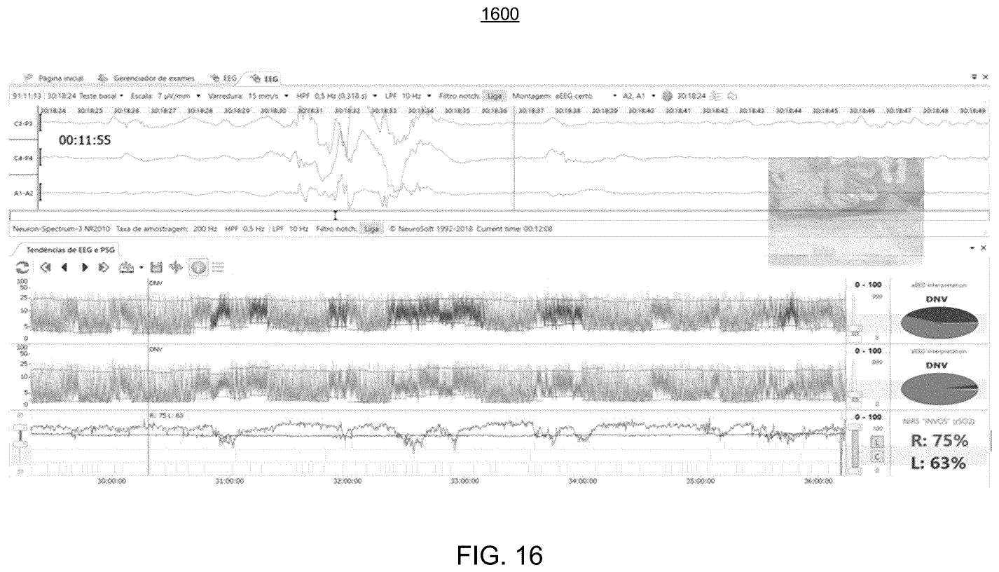

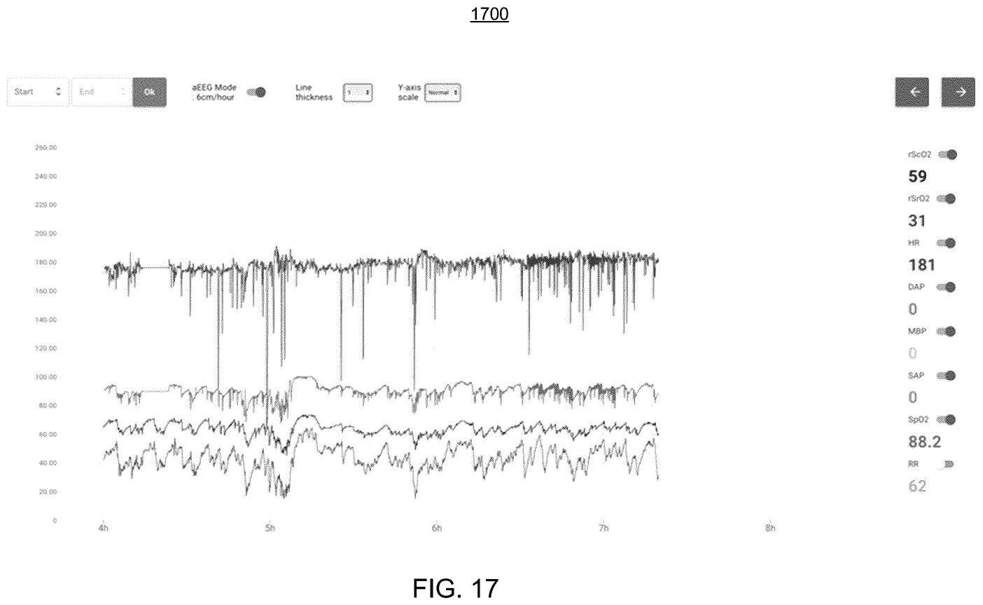

FIG. 15 is an example GUI 1500 illustrating a tracing of a preterm infant with 25 weeks of gestational age on the second day of life with compromised autoregulation. The GUI 1500 shows a high coherence index between rScO2 and rSrO2. FIG. 16 is an example GUI 1600 illustrating tracing of a preterm infant, with 25 week of gestational age in the third day of life with intact autoregulation. The GUI 1600 shows a low coherence index between rScO2 and rSrO2. FIG. 17 is an example GUI 1700 illustrating tracing of a preterm infant, with 23 weeks of gestational age in the second day of life with compromised autoregulation.

In some embodiments, the software combines clinical and multiparametric data to dynamically assess the risk of infants having clinical deterioration and brain injury. The assessed risk is indicated by an overall risk score.

Early detection of subacute, potentially catastrophic illness using available data is a clinical imperative. The overall risk score can indicate a risk of imminent events in real time. Patients deteriorate for a variety of reasons, and it is unlikely that a single predictor, such as abnormal blood pressure or heart rate, will allow quick diagnosis and prescribe treatment interventions. The overall risk score described herein is better than warning scores generated based on comparing vital signs and laboratory tests. Laboratory tests require blood draws and time to process.

In some embodiments, the overall risk score is generated based on a combination of data collected automatically from multi-modality monitoring and data collected with clinically relevant information. In some embodiments, a first score (also referred to as an automatic score) is generated based on data collected automatically from muti-modality monitoring, e.g., near real time information from multiple monitors (e.g., EEG, NIRS, and vital signs). A second score (also referred to as clinical score) is generated based on clinically relevant information.

The automatic score is generated based on analyzing different combinations of subtle changes in each data stream, such as heart rate, rScO2, rSrO2, SpO2, MBP. In some embodiments, each abnormal parameter is measured according to its severity to generate an automatic score. A weight is assigned to each abnormal parameter, and an overall automatic score is generated based on multiple weighted automatic scores. In some embodiments, the weight is determined based on analysis of historical data using machine learning.

The clinical score is generated based on clinical information (e.g., GA, BW, ventilatory support, use of medications, etc.) that is input in the system. In some embodiments, each of multiple clinical parameters is used to generate a clinical score, a weight is assigned to each clinical score, and an overall clinical score is generated based on the multiple weighted clinical scores.

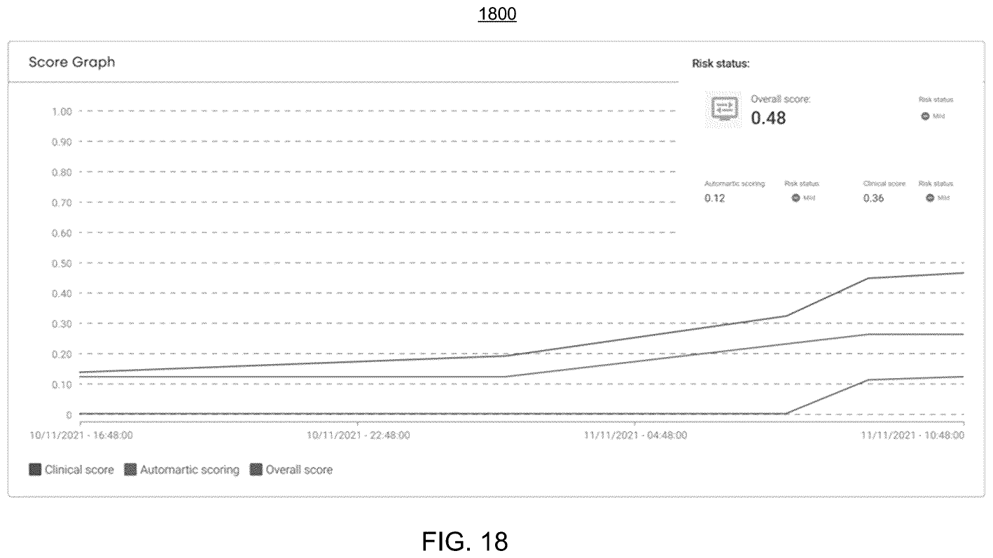

The overall score values (including the overall score, the automatic score, and/or the clinic score) may be displayed in a trendline and time-synced manner with other monitoring data received from the patient, which may inform and guide clinicians at the bedside based on how the patient progress in the ICU, and determine whether additional intervention is needed. FIG. 18 is an example GUI displaying trendlines of three score values, including the overall scores, the automatic scores, and the clinic scores.

### Example Method of Multi-Modality Monitoring of Infants

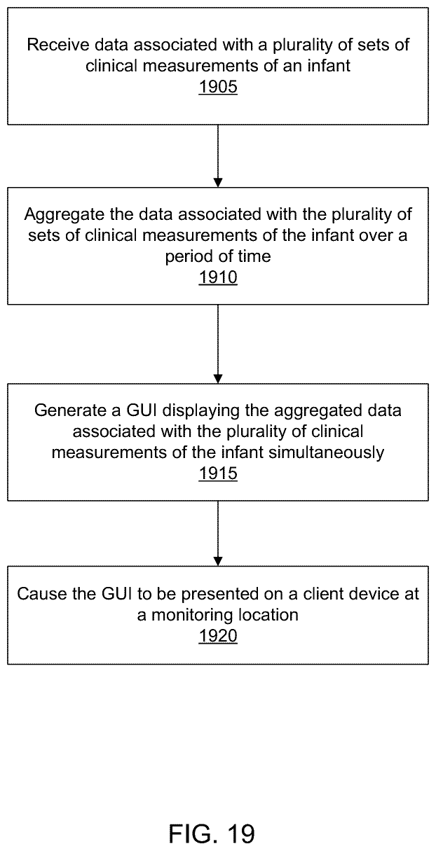

FIG. 19 is a flowchart of a method 1900 of multi-modality monitoring of infants. The method 1900 may be performed by the remote monitoring server 102 or any computing system that is capable of receiving infant monitoring data from infant care centers and processing received data. Alternatively, the method 1900 may be performed by a combination of the remote monitoring server 102, local monitoring system 114, and/or infant monitoring device 112. In various embodiments, the method includes different or additional steps than those described in conjunction with FIG. 19. Further, in some embodiments, the steps of the method may be performed in different orders than the order described in conjunction with FIG. 19.

The remote monitoring server 102 receives 1905 data associated with a plurality of sets of clinical measurements of an infant. The infant is being monitored at an infant care facility that is remote from the remote monitoring server 102. There may be many different infant care facilities, each of which has one or more infants being monitored. The remote monitoring server 102 may receive data associated with each of the monitored infant simultaneously.

The remote monitoring server 102 aggregates 1910 the data associated with the plurality of sets of clinical measurements of the infant over a period of time. The remote monitoring server 102 generates 1915 a GUI displaying the aggregated data associated with the plurality of sets of clinical measurements of the infant simultaneously. The remote monitoring server 102 causes 1920 the GUI to be presented on a client device at a monitoring facility. For example, multiple monitoring devices may be coupled to the infant, such as an aEEG monitor, an NIRS monitor, and a heart rate monitor. The multiple monitoring devices simultaneously perform clinical measurements. Each monitoring device generates a set of clinical measurement over time at a same or different frequency. The remote monitoring server 102 is configured to time-synchronize the multiple sets of clinical measurement, and cause the multiple sets of clinical measurement to be displayed on the GUI in a time-synchronized manner, as shown in FIGS. 4A, 4B.

### Example Machine Learning Models

In various embodiments, a wide variety of machine learning techniques may be used. Examples include different forms of supervised learning, unsupervised learning, and semi-supervised learning such as decision trees, support vector machines (SVMs), regression, Bayesian networks, and genetic algorithms. Deep learning techniques such as neural networks, including convolutional neural networks (CNN), recurrent neural networks (RNN) and long short-term memory networks (LSTM), may also be used. For example, various object recognitions performed by visual reference engine 240, localization, recognition of objects and particularly thin objects, and other processes may apply one or more machine learning and deep learning techniques.

In various embodiments, the training techniques for a machine learning model may be supervised, semi-supervised, or unsupervised. In supervised learning, the machine learning models may be trained with a set of training samples that are labeled. For example, for a machine learning model trained to classify objects, the training samples may be different pictures of objects labeled with the type of objects. The labels for each training sample may be binary or multi-class. In training a machine learning model for image segmentation, the training samples may be pictures of regularly shaped objects in various storage sites with segments of the images manually identified. In some cases, an unsupervised learning technique may be used. The samples used in training are not labeled. Various unsupervised learning technique such as clustering may be used. In some cases, the training may be semi-supervised with training set having a mix of labeled samples and unlabeled samples.

A machine learning model may be associated with an objective function, which generates a metric value that describes the objective goal of the training process. For example, the training may intend to reduce the error rate of the model in generating predictions. In such a case, the objective function may monitor the error rate of the machine learning model. In object recognition (e.g., object detection and classification), the objective function of the machine learning algorithm may be the training error rate in classifying objects in a training set. Such an objective function may be called a loss function. Other forms of objective functions may also be used, particularly for unsupervised learning models whose error rates are not easily determined due to the lack of labels. In image segmentation, the objective function may correspond to the difference between the model's predicted segments and the manually identified segments in the training sets. In various embodiments, the error rate may be measured as cross-entropy loss, L1 loss (e.g., the sum of absolute differences between the predicted values and the actual value), L2 loss (e.g., the sum of squared distances).

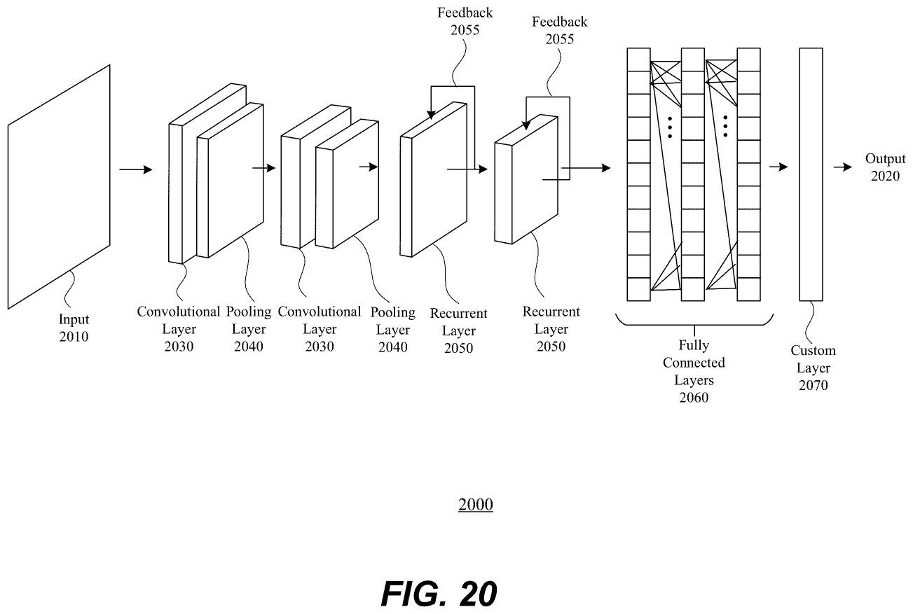

Referring to FIG. 20, a structure of an example CNN is illustrated, according to an embodiment. The CNN 2000 may receive an input 2010 and generate an output 2020. The CNN 2000 may include different kinds of layers, such as convolutional layers 2030, pooling layers 2040, recurrent layers 2050, full connected layers 2060, and custom layers 2070. A convolutional layer 2030 convolves the input of the layer (e.g., an image) with one or more kernels to generate different types of images that are filtered by the kernels to generate feature maps. Each convolution result may be associated with an activation function. A convolutional layer 2030 may be followed by a pooling layer 2040 that selects the maximum value (max pooling) or average value (average pooling) from the portion of the input covered by the kernel size. The pooling layer 2040 reduces the spatial size of the extracted features. In some embodiments, a pair of convolutional layer 2030 and pooling layer 2040 may be followed by a recurrent layer 2050 that includes one or more feedback loop 2055. The feedback 2055 may be used to account for spatial relationships of the features in an image or temporal relationships of the objects in the image. The layers 2030, 2040, and 2050 may be followed in multiple fully connected layers 2060 that have nodes (represented by squares in FIG. 20) connected to each other. The fully connected layers 2060 may be used for classification and object detection. In one embodiment, one or more custom layers 2070 may also be presented for the generation of a specific format of output 2020. For example, a custom layer may be used for image segmentation for labeling pixels of an image input with different segment labels.

The order of layers and the number of layers of the CNN 2000 in FIG. 20 is for example only. In various embodiments, a CNN 2000 includes one or more convolutional layer 2030 but may or may not include any pooling layer 2040 or recurrent layer 2050. If a pooling layer 2040 is present, not all convolutional layers 2030 are always followed by a pooling layer 2040. A recurrent layer may also be positioned differently at other locations of the CNN. For each convolutional layer 2030, the sizes of kernels (e.g., 3×3, 5×5, 7×7, etc.) and the numbers of kernels allowed to be learned may be different from other convolutional layers 2030.

A machine learning model may include certain layers, nodes, kernels and/or coefficients. Training of a neural network, such as the CNN 2000, may include forward propagation and backpropagation. Each layer in a neural network may include one or more nodes, which may be fully or partially connected to other nodes in adjacent layers. In forward propagation, the neural network performs the computation in the forward direction based on outputs of a preceding layer. The operation of a node may be defined by one or more functions. The functions that define the operation of a node may include various computation operations such as convolution of data with one or more kernels, pooling, recurrent loop in RNN, various gates in LSTM, etc. The functions may also include an activation function that adjusts the weight of the output of the node. Nodes in different layers may be associated with different functions.

Each of the functions in the neural network may be associated with different coefficients (e.g. weights and kernel coefficients) that are adjustable during training. In addition, some of the nodes in a neural network may also be associated with an activation function that decides the weight of the output of the node in forward propagation. Common activation functions may include step functions, linear functions, sigmoid functions, hyperbolic tangent functions (tanh), and rectified linear unit functions (ReLU). After an input is provided into the neural network and passes through a neural network in the forward direction, the results may be compared to the training labels or other values in the training set to determine the neural network's performance. The process of prediction may be repeated for other images in the training sets to compute the value of the objective function in a particular training round. In turn, the neural network performs backpropagation by using gradient descent such as stochastic gradient descent (SGD) to adjust the coefficients in various functions to improve the value of the objective function.

Multiple rounds of forward propagation and backpropagation may be performed. Training may be completed when the objective function has become sufficiently stable (e.g., the machine learning model has converged) or after a predetermined number of rounds for a particular set of training samples. The trained machine learning model can be used for performing prediction, object detection, image segmentation, or another suitable task for which the model is trained.

In various embodiments, the training samples described above may be refined and continue to re-train the model, which the model's ability to perform the inference tasks. In some embodiments, this training and re-training processes may repeat, which results in a computer system that continues to improve its functionality through the use-retraining cycle. For example, after the model is trained, multiple rounds of re-training may be performed. The process may include periodically retraining the machine learning model. The periodic retraining may include obtaining an additional set of training data, such as through other sources, by usage of users, and by using the trained machine learning model to generate additional samples. The additional set of training data and later retraining may be based on updated data describing updated parameters in training samples. The process may also include applying the additional set of training data to the machine learning model and adjusting parameters of the machine learning model based on the applying of the additional set of training data to the machine learning model. The additional set of training data may include any features and/or characteristics that are mentioned above. The retraining may be triggered by any retraining criteria that are discussed in this disclosure, such as a predictability or a loss fuction having value that is below a threshold value.

In some embodiments, model distillation may be used to transfer knowledge from a trained model to another model with reduced complexity while maintaining similar predictive performance. A trained model, often referred to as a teacher model, may generate outputs such as logits, confidence scores, or probability distributions over possible predictions. These outputs may serve as soft labels for training a student model, which typically has fewer parameters or a more efficient architecture. The student model may learn to approximate the teacher model's decision boundary by minimizing the difference between its own predictions and the teacher model's outputs. This process may involve loss functions such as Kullback-Leibler (KL) divergence or cross-entropy loss with temperature scaling to ensure that the student model effectively captures the patterns and representations learned by the teacher model.

In some embodiments, model distillation may be performed using different techniques depending on the level of knowledge transfer between the teacher and student models. One common approach is logit-based distillation, where the student model learns from the soft probability distributions produced by the teacher model instead of hard class labels. The temperature parameter in the softmax function may be adjusted to smooth the probability distribution, allowing the student model to capture subtle relationships between classes. Another approach is feature-based distillation, where intermediate feature representations from the teacher model are used to guide the student model's learning process. In this method, layer activations, embeddings, or attention maps from the teacher model may be matched with corresponding layers in the student model to enforce structural similarity. Additionally, response-based distillation may be used, where the teacher model's decisions, such as classification outputs or ranking scores, are directly used to supervise the student model. In some cases, hybrid approaches may combine multiple distillation techniques to optimize both predictive accuracy and model efficiency. By selecting an appropriate distillation strategy, a system may tailor knowledge transfer to specific tasks and deployment constraints.

In some embodiments, model distillation may be applied in scenarios where computational efficiency is a priority, such as deploying models on edge devices, mobile applications, or cloud environments with resource constraints. The distilled student model may inherit the generalization ability of the teacher model while reducing memory footprint and inference latency. In some cases, multiple teacher models may be used to provide diverse outputs, allowing the student model to integrate knowledge from different sources. The distillation process may also incorporate additional regularization techniques, such as weight pruning or quantization, to further optimize the student model's efficiency. By leveraging model distillation, a system may achieve a balance between model performance and computational efficiency, enabling scalable deployment across various machine learning applications.

### Computing Machine Architecture

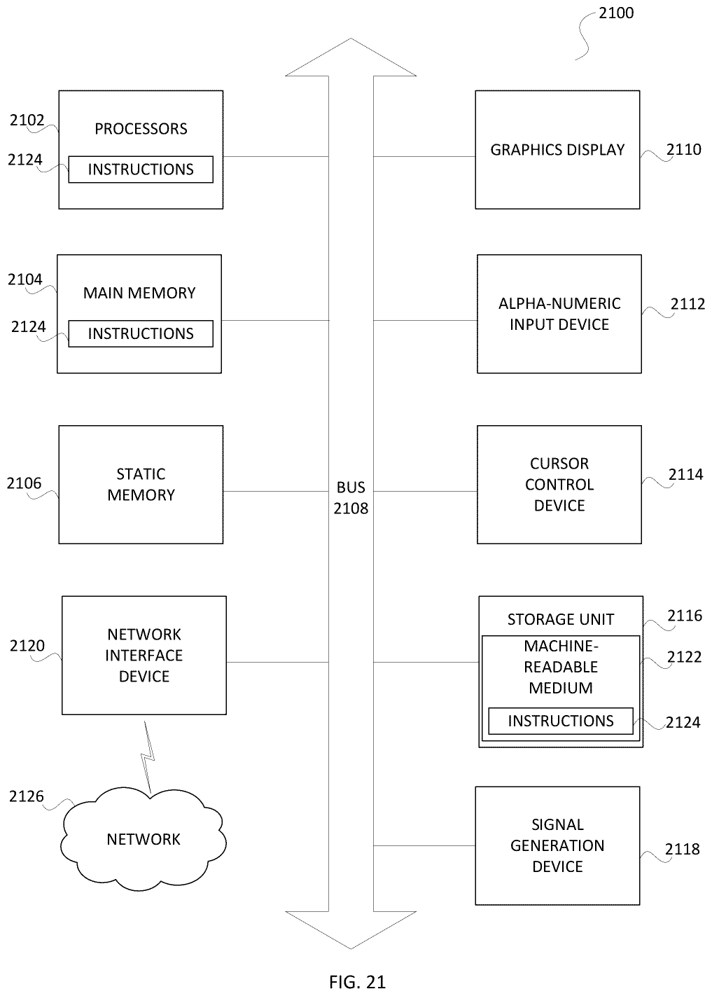

FIG. 21 is a block diagram illustrating components of an example computing machine that is capable of reading instructions from a computer-readable medium and execute them in a processor (or controller). A computer described herein may include a single computing machine shown in FIG. 21, a virtual machine, a distributed computing system that includes multiple nodes of computing machines shown in FIG. 21, or any other suitable arrangement of computing devices.

By way of example, FIG. 21 shows a diagrammatic representation of a computing machine in the example form of a computer system 2100 within which instructions 2124 (e.g., software, source code, program code, expanded code, object code, assembly code, or machine code), which may be stored in a computer-readable medium for causing the machine to perform any one or more of the processes discussed herein may be executed. In some embodiments, the computing machine operates as a standalone device or may be connected (e.g., networked) to other machines. In a networked deployment, the machine may operate in the capacity of a server machine or a client machine in a server-client network environment, or as a peer machine in a peer-to-peer (or distributed) network environment.

The structure of a computing machine described in FIG. 21 may correspond to any software, hardware, or combined components shown in FIGS. 1-3, including but not limited to, the remote monitoring server 102, local monitoring system 114, infant monitoring device 112, expert client device 122, 132, and various engines, interfaces, terminals, and machines shown in FIG. 2. While FIG. 21 shows various hardware and software elements, each of the components described in FIGS. 1 and 2 may include additional or fewer elements.

By way of example, a computing machine may be a personal computer (PC), a tablet PC, a set-top box (STB), a personal digital assistant (PDA), a cellular telephone, a smartphone, a web appliance, a network router, an internet of things (IoT) device, a switch or bridge, or any machine capable of executing instructions 2124 that specify actions to be taken by that machine. Further, while only a single machine is illustrated, the term “machine” and “computer” may also be taken to include any collection of machines that individually or jointly execute instructions 2124 to perform any one or more of the methodologies discussed herein.

The example computer system 2100 includes one or more processors 2102 such as a CPU (central processing unit), a GPU (graphics processing unit), a TPU (tensor processing unit), a DSP (digital signal processor), a system on a chip (SOC), a controller, a state equipment, an application-specific integrated circuit (ASIC), a field-programmable gate array (FPGA), or any combination of these. Parts of the computing system 2100 may also include a memory 2104 that store computer code including instructions 2124 that may cause the processors 2102 to perform certain actions when the instructions are executed, directly or indirectly by the processors 2102. Instructions can be any directions, commands, or orders that may be stored in different forms, such as equipment-readable instructions, programming instructions including source code, and other communication signals and orders. Instructions may be used in a general sense and are not limited to machine-readable codes. One or more steps in various processes described may be performed by passing through instructions to one or more multiply-accumulate (MAC) units of the processors.

The performance of certain operations may be distributed among more than one processor, not only residing within a single machine, but deployed across a number of machines. In some example embodiments, the one or more processors or processor-implemented modules may be located in a single geographic location (e.g., within a home environment, an office environment, or a server farm). In other example embodiments, one or more processors or processor-implemented modules may be distributed across a number of geographic locations. Even though in the specification or the claims may refer some processes to be performed by a processor, this should be construed to include a joint operation of multiple distributed processors.

The computer system 2100 may include a main memory 2104, and a static memory 2106, which are configured to communicate with each other via a bus 2108. The computer system 2100 may further include a graphics display unit 2110 (e.g., a plasma display panel (PDP), a liquid crystal display (LCD), a projector, or a cathode ray tube (CRT)). The graphics display unit 2110, controlled by the processors 2102, displays a graphical user interface (GUI) to display one or more results and data generated by the processes described herein. The computer system 2100 may also include alphanumeric input device 2112 (e.g., a keyboard), a cursor control device 2114 (e.g., a mouse, a trackball, a joystick, a motion sensor, or other pointing instruments), a storage unit 2116 (a hard drive, a solid-state drive, a hybrid drive, a memory disk, etc.), a signal generation device 2118 (e.g., a speaker), and a network interface device 2120, which also are configured to communicate via the bus 2108.

The storage unit 2116 includes a computer-readable medium 2122 on which is stored instructions 2124 embodying any one or more of the methodologies or functions described herein. The instructions 2124 may also reside, completely or at least partially, within the main memory 2104 or within the processor 2102 (e.g., within a processor's cache memory) during execution thereof by the computer system 2100, the main memory 2104 and the processor 2102 also constituting computer-readable media. The instructions 2124 may be transmitted or received over a network 2126 via the network interface device 2120.

While computer-readable medium 2122 is shown in an example embodiment to be a single medium, the term “computer-readable medium” should be taken to include a single medium or multiple media (e.g., a centralized or distributed database, or associated caches and servers) able to store instructions (e.g., instructions 2124). The computer-readable medium may include any medium that is capable of storing instructions (e.g., instructions 2124) for execution by the processors (e.g., processors 2102) and that cause the processors to perform any one or more of the methodologies disclosed herein. The computer-readable medium may include, but not be limited to, data repositories in the form of solid-state memories, optical media, and magnetic media. The computer-readable medium does not include a transitory medium such as a propagating signal or a carrier wave.

### Additional Considerations

The foregoing description of the embodiments of the invention has been presented for the purpose of illustration; it is not intended to be exhaustive or to limit the invention to the precise forms disclosed. Persons skilled in the relevant art can appreciate that many modifications and variations are possible in light of the above disclosure.

Some portions of this description describe the embodiments of the invention in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. In one embodiment, a software module is implemented with a computer program product comprising a computer-readable medium containing computer program code, which can be executed by a computer processor for performing any or all of the steps, operations, or processes described.

Embodiments of the invention may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a tangible computer readable storage medium, which include any type of tangible media suitable for storing electronic instructions and coupled to a computer system bus. Furthermore, any computing systems referred to in the specification may include a single processor or may be architectures employing multiple processor designs for increased computing capability.

Embodiments of the invention may also relate to a computer data signal embodied in a carrier wave, where the computer data signal includes any embodiment of a computer program product or other data combination described herein. The computer data signal is a product that is presented in a tangible medium or carrier wave and modulated or otherwise encoded in the carrier wave, which is tangible, and transmitted according to any suitable transmission method.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention, which is set forth in the following claims.

What is claimed is:

1. A method comprising:
receiving, at a remote server that is geographically remote from an infant care location where an infant is being monitored, real-time data associated with a plurality of sets of clinical measurements of the infant, the plurality of sets of clinical measurements including a vital measurement of the infant transmitted from a sensor physically attached to the infant;
storing the real-time data associated with the plurality of sets of clinical measurements of the infant as a plurality of time series data in a data store associated with the remote server;
causing a graphical user interface to present the plurality of time series data in a time-synchronized manner on a monitor located at a monitoring facility, the plurality of time series data displayed in real-time as the plurality of sets of clinical measurements are measured at the infant care location, wherein the monitoring facility is located geographically remotely from the infant care location;
receiving a communication from the monitoring facility that the infant is at risk of a potential brain injury condition; and
providing a notification to the infant care location regarding the potential brain injury condition.

2. The method of claim 1, wherein the GUI displays a list of a plurality of monitored infants.

3. The method of claim 1, wherein the GUI displays an overview of a number of registered infant care facilities and a number of monitored infants at the number of registered infant care facilities.

4. The method of claim 1, wherein the GUI displays a number of monitored infants with a particular clinical condition.

5. The method of claim 4, wherein the particular clinical condition is seizure.

6. The method of claim 1, wherein the GUI displays a number of open tickets indicating communications between users at the plurality of infant care facilities and users at the monitoring facility.

7. The method of claim 1, further comprising:
classifying monitored infants into a plurality of risk status;
color coding the plurality of risk status into a plurality of colors; and
displaying the monitored infants based on the color coding.

8. The method of claim 1, further comprising:
detecting an anomaly associated with a clinical condition based on the aggregated data; and
responsive to detecting the anomaly, generating and sending an alert at a client device at the monitoring facility.

9. The method of claim 8, further comprising:
sending the alert to a client device at the infant care facility.

10. The method of claim 8, wherein detecting an anomaly comprises:
accessing a machine-learning model trained on training datasets, training datasets comprising historical data associated with clinical measurements of a plurality of infants;
applying the data associated with the plurality of sets of clinical measurements of the infant to the machine-learning model, causing the machine-learning model to determine a score indicating a likelihood of the anomaly being present; and
responsive to determining that the score is greater than a threshold, determining that the anomaly is present.

11. The method of claim 10, wherein the machine-learning model is trained based on an unsupervised training method.

12. The method of claim 10, wherein the training datasets include a plurality of time series, each of which is associated with a timeframe of data associated with clinical measurements of an infant among the plurality of infants, and the machine-learning model is a classifier trained based on contrastive learning.

13. The method of claim 12, wherein the timeframe is no less than 10 seconds.

14. The method of claim 12, further comprising:
receiving a user input indicating a timeframe value; and
responsive to receiving the user input, setting the timeframe to the timeframe value.

15. The method of claim 12, further comprising:
computing an average value of a set of clinical measurements during a timeframe; and
visualizing the average value on the GUI.

16. The method of claim 10, the machine-learning model is trained to receive as input a time series associated with a time frame of data associated with clinical measurement of the infant, to output a determination of whether the infant is having a seizure.

17. The method of claim 10, wherein the machine-learning model is trained to output a score indicating a probability of the infant capable of cerebral autoregulation.

18. The method of claim 10, wherein the machine-learning model is trained to output a score indicating an overall risk of the infant having clinical deterioration or brain injury.

19. The method of claim 10, wherein the machine-learning model is trained to:
output a first score based on data associated with the plurality of sets of clinical measurements of the infant;
output a second score based on clinical information including support or mediation being received by the infant; and
output a third score indicating an overall risk of the infant having clinical deterioration or brain injury based on the first score and the second score.

20. The method of claim 19, wherein the first score, second score, and third score are generated repeatedly at different times, and the GUI further displays the first score, the second score, or the third score at different times to show risk trend of the infant.
